(19) United States
(12) Patent Application Publication
(10) Pub. No.: US 2017/0065667 A1
(43) Pub. Date: Mar. 9, 2017

(54) METHODS FOR SYNCHRONIZATION OF ESTROUS CYCLES AND OVULATION FOR FIXED TIME ARTIFICIAL INSEMINATION IN CATTLE

(71) Applicant: Parnell Technologies Pty Ltd., Alexandria (AU)

(72) Inventors: Robert Thomas JOSEPH, Kansas City, MO (US); Fenella COCHRANE, Kansas City, MO (US); Milo Charles WILTBANK, Verona, WI (US)

(21) Appl. No.: 14/699,904

(22) Filed: Apr. 29, 2015

Related U.S. Application Data
(60) Provisional application No. 61/986,021, filed on Apr. 29, 2014.

(51) Int. Cl. A61K 38/09; A61D 19/02; A61K 31/5575

(52) U.S. Cl. CPC A61K 38/09; A61K 31/5575; A61D 19/02

(57) ABSTRACT

The present application relates to the reproductive management of cattle, and more particularly, to the synchronization of estrous cycles and ovulation for fixed time artificial insemination of cattle with the addition of a second prostaglandin treatment in Ovsynch-type protocols resulting in the complete regression of the corpus luteum and an increase in pregnancies per artificial insemination in multiparous cows.

### CROSS-REFERENCE TO RELATED APPLICATIONS

This application claims priority in U.S. Provisional Application No. 61/986,021, filed Apr. 29, 2014, the contents of which are hereby incorporated by reference.

### BACKGROUND

The present disclosed subject matter relates generally to reproductive management of cattle, and more particularly to the synchronization of estrous cycles and ovulation for fixed time artificial insemination of cattle.

Managing the reproductive cycle of cattle is an important variable in maximizing the profitability of the beef cow or dairy cow operator. The reproductive cycle of domestic animals is controlled by hormones. The exogenous administration of hormones allows an operator to synchronize the estrous cycle and manage the time of ovulation of the cattle within their herd. Artificial insemination (AI) of cattle in conjunction with management of the time of ovulation of an ovum allows an operator to manage the timing of labor, genetics, birth of new offspring and improve reproductive efficiency of the herd.

The pituitary gland is an endocrine gland that secretes hormones, composed of an anterior and posterior lobe, located at the base of the brain. Endogenous gonadotropins are produced by the anterior lobe in response to gonadotropin-releasing hormone (GnRH) produced by the hypothalamus, and estrogen produced by follicles of the ovaries. Gonadotropins include follicle stimulating hormone (FSH) and luteinizing hormone (LH). Production of FSH and LH are regulated by the circulating ovarian hormones such as estradiol-1713 (E2) from the ovarian follicle and progesterone (P4) produced by the corpus luteum (CL). FSH and LH circulating in the blood have an effect upon the ovaries that relate to the morphological changes leading to ovulation and, if conception occurs, pregnancy of the cow.

The reproductive or estrous cycle of cattle is generally 21 days long, and generally includes two phases, a follicular phase followed by a longer luteal phase. The period between the end of the follicular phase and the beginning of the luteal phase is when ovulation occurs. Natural breeding by a bull or artificial insemination (AI) needs to occur as close to the time of ovulation to ensure that a viable sperm can bind to a viable ovum thus increasing the chance of successful fertilization. To facilitate the optimal timing of AI in a group of cows that require breeding, operators can use exogenous reproductive hormones administered in specified sequence and timings to cause a group of cows to ovulate. In cows with synchronized ovulation, all cows can be inseminated at a known time that is determined by the operator to allow efficient use of resources, a process termed Fixed Timed Artificial Insemination (FTAI). Thus, the use of synchronization of ovulation can provide more reliable timing of AI, in relation to ovulation in a group of cattle that are available for breeding.

During the follicular phase, cells within the ovary undergo various changes to produce a viable ovum that is capable of ovulation, fertilization, and production of an embryo. In particular, the granulosa cells within the follicle contain receptors for FSH and in response to circulating FSH, these cells proliferate and the follicle grows. Thus, the presence of FSH stimulates the growth and maturation of responsive follicles. A maturing follicle generates an increasing amount of estrogen as the ovum grows and matures, acquiring the cellular properties that are essential for fertility. There are also increasing layers of granulosa cells in the follicle that are essential for hormone production and for producing factors that are essential for the maturation of the ovum.

Various behavioral changes are characteristic of the “heat” or period of estrus which immediately precedes ovulation and can act as a detectable sign that a cow is ready to be inseminated. The onset of estrus is induced by an increase in circulating estradiol-17β (E2) concentrations. The increase in E2 also induces a dramatic increase in secretion of GnRH from neurons in the hypothalamus. This increase in GnRH causes an increase in secretion of LH from the pituitary gland, also known as the LH surge. The LH surge induces ovulation of the follicle. The timing of ovulation is naturally synchronized with behavioral estrus because the LH surge and estrus are both induced by an elevation in E2 concentrations in the presence of low circulating P4 concentrations. Ovulation occurs approximately 24-32 hours after the onset of estrus. Once ovulation occurs and the ovum is released, the layers of cells that remain from the ovulated follicle differentiate into a structure called the corpus luteum (CL). Thus, the LH surge not only induces ovulation but also causes differentiation of the follicular cells into the distinctive cells that are present in the CL, termed luteal cells. Once formed, the CL produces P4 that acts as an inhibitory agent on the hypothalamus, decreasing production of GnRH, thereby inhibiting pulses of LH and the LH surge and thereby inhibiting the final stages of preovulatory follicle development. Further, it is the production of P4 by the CL that causes changes in the uterus that allows development of the early embryo, attachment of the embryo to the uterus, and maintenance of the pregnancy.

After a period of time of influence of P4 from the CL, if a pregnancy has not occurred, the uterus will begin releasing prostaglandin F2α (PGF2α) which causes a decrease in function of the CL and eventually death of the luteal cells, a process termed CL regression, or luteolysis. Regression of the CL causes a rapid decrease in circulating P4 which removes the inhibition of GnRH by the hypothalamus. This marks the commencement of a new estrous cycle leading to the next ovulation which provides a new opportunity for a cow to become pregnant. Specifically, the removal of the P4 inhibition (by luteolysis of the CL) allows the recommencement of FSH secretion by the pituitary gland which triggers the commencement of the Follicular phase. It is critical that complete CL regression occurs for continuation of the next estrous cycle. Cows without complete CL regression have greatly reduced fertility. Thus, a key to artificially controlling the time of ovulation is completely removing the influence of P4 by complete regression of the CL. It is critical that the concentration of P4 in the blood is very low, for normal maturation of the ovarian follicle, ovulation of the follicle, and normal transport of sperm and the ovum in the reproductive tract so that fertilization will occur. Even small elevations in circulating P4 near the time of ovulation will cause disruption in these processes and greatly reduce fertility.

One approach to precisely managing reproduction in beef and dairy cattle is known as the Ovsynch (Ovulation Synchronization) program. A traditional Ovsynch program employs the exogenous administration of hormones in a specific sequence in order to precisely synchronize circulating concentrations of ovarian hormones and precisely synchronize the time of ovulation in lactating dairy cattle or beef cattle. By precisely synchronizing the time of ovulation through a specific series of hormonal treatments, the day of first AI for a group of cattle can be selected by the operator. Thus, use of Ovsynch allows the operator to perform FTAI. Such an Ovsynch program begins with a first administration of a GnRH composition to ovulate an existing dominant follicle and initiate a new follicular wave, followed by administration of a PGF2α composition to initiate regression of any CL that are present on the ovary. The typical Ovsynch program then utilizes a second administration of a GnRH composition just prior to AI to cause ovulation of the dominant follicle and thus produce synchronized ovulation. There are two different analogs of PGF2α that are typically administered for regression of the CL in an Ovsynch program, dinoprost and cloprostenol.

Reproductive efficiency in lactating dairy cows is suboptimal, due to inadequate service risk and reduced pregnancies per AI (P/AI).^{19, 28, 31} Timed AI programs have been developed to increase the service risk, however, fertility is still suboptimal when uncomplicated protocols, such as Ovsynch, are utilized.^{21, 22, 23} Fertility to the Ovsynch protocol is reduced due to lack of proper synchronization of follicular and luteal function and suboptimal hormonal concentrations in some cows during the protocol.^{10, 29, 32} Initiation of Ovsynch at more optimal stages of the estrous cycle, such as day 6 or 7, increases synchronization to the protocol and increases fertility.^{30, 32} In order to practically increase the percentage of cows at an optimal stage of the cycle at the initiation of Ovsynch, presynchronization protocols have been developed such as Presynch-Ovsynch^{16, 6, 18} Double-Ovsynch^{1, 13, 26} and G-6-G.^{2, 20} These programs increase fertility primarily because of increased synchronization of ovarian function and circulating hormone concentrations during Ovsynch.^{12, 32, 33}

Presynchronization protocols that result in improved fertility during timed AI protocols generally result in increased ovulation to the first GnRH of Ovsynch.^{7, 10, 32} The newly-formed CL due to this ovulation can increase circulating P4 during the preovulatory follicle growth phase, potentially increasing fertility and reducing double ovulation rate.^{3, 9, 33} Indeed, cows that ovulate to the first GnRH treatment generally have greater fertility than cows that do not ovulate.^{2, 5, 14} However, the new CL, induced by the first GnRH treatment of Ovsynch, may be difficult to regress with a single treatment with PGF2α. Lack of complete regression of the CL to the PGF2α treatment has been observed in 10 to 25% of cows treated with Ovsynch.^{4, 15, 32} In these studies, cows that have small elevations in circulating P4 near AI, due to inadequate CL regression, have reduced fertility. This is particularly important if a shortened protocol is utilized, like the 5-day protocol, because the very young CL (present if ovulation to the first GnRH occurs) is difficult to regress with a single PGF2α treatment.^{17, 24, 25} However, even in the 7 day Ovsynch protocol, lack of regression of the CL is an important problem, leading to reductions in fertility during Ovsynch or modifications of Ovsynch protocols.^{12, 15, 33} As would be expected, incomplete CL regression is more likely in cows with a new CL than in cows that did not ovulate to the first GnRH treatment.¹²

In order to overcome the problem with inadequate CL regression during timed AI protocols, two general strategies have been utilized. One strategy is to give an increased dose of PGF2α during either the 5 day or 7 day Ovsynch protocol.^{10, 24} A second strategy is to give a second dose of PGF2α on the subsequent day after the first PGF2α treatment of a 5 day or 7 day Ovsynch protocol. In the 5 day Ovsynch protocol, fertility was reduced if only a single PGF2α was given²⁵ or if two PGF2α treatments were given on the same day (5 days after GnRH) compared to giving one treatment on day 5 and a second on day 6.²⁴ However, in the 7 day protocol, increasing the dose of cloprostenol from 500 to 750 μg increased CL regression in multiparous but not primiparous cows and tended to increase fertility.¹⁰ Another study using the 7 day Ovsynch protocol, compared CL regression and fertility in cows treated with one PGF2α (day 7) or two PGF2α treatments (day 7 and day 8).⁴ There was an increased percentage of cows with complete CL regression (<0.4 ng/mL 56 hours after PGF2α) after two (326/341=95.6%) compared to one (301/356=84.6%) PGF2α treatment. However, no improvement in fertility was detectable, statistically. This study was probably statistically underpowered (n=772), since the 5.7% improvement in fertility that was observed in first service cows (47.0 to 52.7%) was of the expected magnitude but was not statistically significant.⁴

A drawback to the traditional Ovsynch program is a sub optimal fertility at FTAI. One factor that is reducing fertility is the presence of progesterone near the time of AI due to a lack of complete regression of the CL.

### SUMMARY

The specific timing of the administration of a second PGF2α composition in an Ovsynch protocol affects the synchronization of the estrous cycle of lactating dairy and beef cattle, improving pregnancy rates following FTAI over traditional Ovsynch protocols. The second administration of a PGF2α composition comprising cloprostenol sodium composition following a first administration of a PGF2α composition reduced the number of cows with inadequate regression of the CL at the time of final GnRH treatment, resulting in increased pregnancies following FTAI in multiparous cows.

### BRIEF DESCRIPTION OF THE DRAWINGS

The drawings constitute a part of this specification and include exemplary embodiments of the disclosed subject matter and illustrate various objects and features thereof.

FIG. 1 shows the relationship between circulating P4 near AI and the percentage of cows that are pregnant after a single AI (conception rate, %) in lactating dairy cows that received Ovsynch.

FIG. 2 is a schematic diagram of the experimental protocol used for Experiments 1 and 2.

FIG. 3 shows the comparison of effect of one vs. two cloprostenol sodium injections on the distribution of cows by circulating P4 at the time of final GnRH treatment.

FIG. 4A shows the relationship between circulating P4 concentrations at the time of the initial cloprostenol sodium treatment on CL regression in cows receiving one vs. two cloprostenol sodium treatments.

FIG. 4B shows the relationship between circulating P4 concentrations at the time of the initial cloprostenol sodium treatment on P/AI in cows receiving one vs. two cloprostenol sodium treatments.

### DETAILED DESCRIPTION

The present disclosed subject matter is based on the surprise finding that a variety of variables, not only the specific timing of the administration of a gonadotropin releasing hormone (GnRH) composition and a prostaglandin F2α (PGF2α) composition, affects the synchronization of the estrous cycle of lactating dairy and beef cattle, thereby affecting pregnancy rates following fixed time artificial insemination (FTAI).

The method of the present disclosed subject matter resulted from the discovery that a second dose of a PGF2α composition subsequent to a first dose of a PGF2α composition is effective in increasing the number of cattle presenting complete regression of the corpus luteum (CL), thereby decreasing circulating blood progesterone (P4) concentrations, and increasing fertility at FTAI.

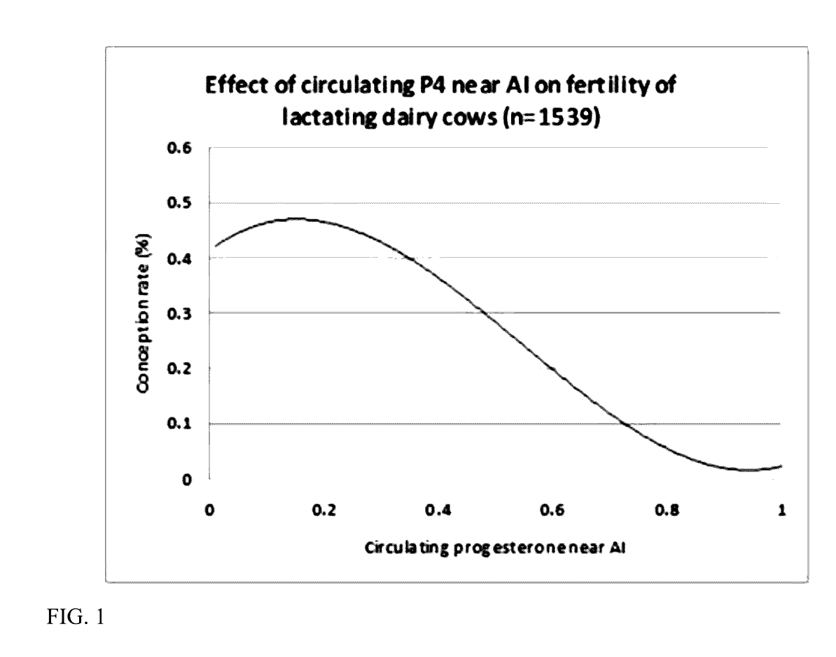

Data indicate that even cows with slight elevations in circulating P4 (>0.1 ng/mL) have large decreases in fertilization of oocytes. The cows with slight elevations in circulating P4 concentrations have greatly reduced fertility (percentage of cows pregnant to an AI). The results were analyzed, and the relationship between circulating P4 and fertility in lactating dairy cows is shown in FIG. 1. As P4 concentrations increased near the time of AI, there was a rapid decrease in fertility (conception rate). Cows with more than 0.4 ng/mL of P4 in the blood near the time of AI had much lower fertility than cows with P4 lower than 0.4 ng/mL. The reason for the elevation in P4 near the time of AI is incomplete regression of the CL. Thus, treatment with a second PGF2α treatment increases the percentage of cows with complete CL regression and improves fertility. An improvement in fertility increases the number of cows pregnant at first AI and improves the reproductive efficiency on the dairy farm. Improvement in reproductive efficiency can translate into a substantial improvement in the economic viability of a dairy operation. For example, improvements in reproduction will lead to more cows in the ideal stage of lactation and improvements in milk production. In addition, improved reproduction will allow healthy, high producing dairy cows to be kept in the dairy herd instead of being sold if they do not become pregnant.

### I. Methods for Administration

The method for synchronizing the estrous cycle in cattle includes the steps of administering a first dose of a GnRH composition in an amount effective to stimulate ovarian follicle development. Subsequent to administering a first dose of GnRH, and after a first period of time sufficient for development of a dominant follicle, administering a first dose of a PGF2α composition in an amount effective to initiate CL regression. In an embodiment, the first period of time is between about 0 days and about 10.5 days. In another embodiment, the first period of time is between about 3.5 days and about 8.75 days. In another embodiment, the first period of time is 7 days.

Subsequent to administering a first dose of PGF2α, and after a second period of time sufficient for luteal regression, administering a second dose of a PGF2α composition in an amount effective to produce complete CL regression. In an embodiment, the second period of time is between about 0 hours and about 56 hours. In another embodiment, the second period of time is between about 12 hours and about 30 hours. In another embodiment, the second period of time is 24 hours.

Subsequent to administering a second dose of PGF2α, and after a third period of time sufficient for complete luteal regression, administering a second dose of a GnRH composition in an amount effective to stimulate synchronized ovulation of the follicle. In an embodiment, the third period of time is about 0 hours to about 56 hours. In another embodiment, the third period of time is between about 24 hours and about 40 hours. In another embodiment, the third period of time is 32 hours. In another embodiment, the third period of time is 56 hours.

Subsequent to administering a second dose of GnRH, and after a fourth period of time, breeding the cow by artificial insemination (AI). In an embodiment, the fourth period of time is between about 0 hours and about 24 hours. In another embodiment, the fourth period of time is between about 8 hours and about 20 hours. In another embodiment, the fourth period of time is 16 hours.

In an embodiment, the first dose of GnRH is a gonadorelin acetate composition. In another embodiment, the first dose of gonadorelin acetate composition is a dose containing between about 0 mcg gonadorelin to about 500 mcg gonadorelin. In another embodiment, the first dose of gonadorelin acetate composition is a dose containing between about 100 mcg gonadorelin to about 400 mcg gonadorelin. In another embodiment, the first dose of gonadorelin acetate composition is a dose containing between about 200 mcg gonadorelin to about 300 mcg gonadorelin. In another embodiment, the first dose of gonadorelin acetate composition is a dose containing 100 mcg gonadorelin. In another embodiment, the first dose of gonadorelin acetate composition is a dose containing 50 mcg gonadorelin.

In an embodiment, the first dose of GnRH is a gonadorelin hydrochloride composition. In another embodiment, the first dose of gonadorelin hydrochloride composition is a dose containing between about 0 mcg gonadorelin to about 500 mcg gonadorelin. In another embodiment, the first dose of gonadorelin hydrochloride composition is a dose containing between about 100 mcg gonadorelin to about 400 mcg gonadorelin. In another embodiment, the first dose of gonadorelin hydrochloride composition is a dose containing between about 200 mcg gonadorelin to about 300 mcg gonadorelin. In another embodiment, the first dose of gonadorelin hydrochloride composition is a dose containing 100 mcg gonadorelin. In another embodiment, the first dose of gonadorelin hydrochloride composition is a dose containing 50 mcg gonadorelin.

In an embodiment, the second dose of GnRH is a gonadorelin acetate composition. In another embodiment, the second dose of gonadorelin acetate composition is a dose containing between about 0 mcg gonadorelin to about 500 mcg gonadorelin. In another embodiment, the second dose of gonadorelin acetate composition is a dose containing between about 100 mcg gonadorelin to about 400 mcg gonadorelin. In another embodiment, the second dose of gonadorelin acetate composition is a dose containing between about 200 mcg gonadorelin to about 300 mcg gonadorelin. In another embodiment, the second dose of gonadorelin acetate composition is a dose containing 100 mcg gonadorelin. In another embodiment, the second dose of gonadorelin acetate composition is a dose containing 50 mcg gonadorelin.

In an embodiment, the second dose of GnRH is a gonadorelin hydrochloride composition. In another embodiment, the second dose of gonadorelin hydrochloride composition is a dose containing between about 0 mcg gonadorelin to about 500 mcg gonadorelin. In another embodiment, the second dose of gonadorelin hydrochloride composition is a dose containing between about 100 mcg gonadorelin to about 400 mcg gonadorelin. In another embodiment, the second dose of gonadorelin hydrochloride composition is a dose containing between about 200 mcg gonadorelin to about 300 mcg gonadorelin. In another embodiment, the second dose of gonadorelin hydrochloride composition is a dose containing 100 mcg gonadorelin. In another embodiment, the second dose of gonadorelin hydrochloride composition is a dose containing 50 mcg gonadorelin.

In an embodiment, the first dose of PGF2α is a cloprostenol sodium composition. In another embodiment, the first dose of cloprostenol sodium composition is a dose containing between about 0 mcg cloprostenol to about 1000 mcg cloprostenol. In another embodiment, the first dose of cloprostenol sodium composition is a dose containing between about 100 mcg cloprostenol to about 900 mcg cloprostenol. In another embodiment, the first dose of cloprostenol sodium composition is a dose containing between about 200 mcg cloprostenol to about 800 mcg cloprostenol. In another embodiment, the first dose of cloprostenol sodium composition is a dose containing between about 300 mcg cloprostenol to about 700 mcg cloprostenol. In another embodiment, the first dose of cloprostenol sodium composition is a dose containing between about 400 mcg cloprostenol to about 600 mcg cloprostenol. In another embodiment, the first dose of cloprostenol sodium composition is a dose containing between about 0 mcg cloprostenol to about 200 mcg cloprostenol. In another embodiment, the first dose of cloprostenol sodium composition is a dose containing between about 80 mcg cloprostenol to about 150 mcg cloprostenol. In another embodiment, the first dose of cloprostenol sodium composition is a dose containing about 100 mcg cloprostenol. In another embodiment, the first dose of cloprostenol sodium composition is a dose containing about 526 mcg cloprostenol.

In an embodiment, the first dose of PGF2α is a dinoprost tromethamine composition. In another embodiment, the first dose of dinoprost tromethamine composition is a dose containing between about 0 mg dinoprost to about 50 mg dinoprost. In another embodiment, the first dose of dinoprost tromethamine composition is a dose containing between about 10 mg dinoprost to about 40 mg dinoprost. In another embodiment, the first dose of dinoprost tromethamine composition is a dose containing between about 20 mg dinoprost to about 30 mg dinoprost. In another embodiment, the first dose of dinoprost tromethamine composition is a dose containing about 25 mg dinoprost.

In an embodiment, the second dose of PGF2α is a cloprostenol sodium composition. In another embodiment, the second dose of cloprostenol sodium composition is a dose containing between about 0 mcg cloprostenol to about 1000 mcg cloprostenol. In another embodiment, the second dose of cloprostenol sodium composition is a dose containing between about 100 mcg cloprostenol to about 900 mcg cloprostenol. In another embodiment, the second dose of cloprostenol sodium composition is a dose containing between about 200 mcg cloprostenol to about 800 mcg cloprostenol. In another embodiment, the second dose of cloprostenol sodium composition is a dose containing between about 300 mcg cloprostenol to about 700 mcg cloprostenol. In another embodiment, the second dose of cloprostenol sodium composition is a dose containing between about 400 mcg cloprostenol to about 600 mcg cloprostenol. In another embodiment, the second dose of cloprostenol sodium composition is a dose containing between about 0 mcg cloprostenol to about 200 mcg cloprostenol. In another embodiment, the second dose of cloprostenol sodium composition is a dose containing between about 80 mcg cloprostenol to about 150 mcg cloprostenol. In another embodiment, the second dose of cloprostenol sodium composition is a dose containing about 100 mcg cloprostenol. In another embodiment, the second dose of cloprostenol sodium composition is a dose containing about 526 mcg cloprostenol.

In an embodiment, the second dose of PGF2α is a dinoprost tromethamine composition. In another embodiment, the second dose of dinoprost tromethamine composition is a dose containing between about 0 mg dinoprost to about 50 mg dinoprost. In another embodiment, the second dose of dinoprost tromethamine composition is a dose containing between about 10 mg dinoprost to about 40 mg dinoprost. In another embodiment, the second dose of dinoprost tromethamine composition is a dose containing between about 20 mg dinoprost to about 30 mg dinoprost. In another embodiment, the second dose of dinoprost tromethamine composition is a dose containing about 25 mg dinoprost.

### II. Hormones

The gonadorelin acetate composition contains an analog of GnRH in an amount effective to stimulate a surge in FSH and LH from the pituitary gland. In an embodiment, the gonadorelin acetate composition is GONAbreed®, comprising gonadorelin acetate, available from Parnell Technologies Pty Ltd, Alexandria, Australia. Each mL of the aqueous solution contains 100 mcg of gonadorelin as gonadorelin acetate, 10 mg benzyl alcohol, 7.47 mg sodium chloride, 8.3 mg sodium phosphate monobasic and 4.8 mg sodium phosphate dibasic. In an embodiment, each mL of the aqueous solution contains between about 1 mcg gonadorelin acetate to about 500 mcg gonadorelin acetate.

The cloprostenol sodium composition contains an analog of PGF2α in an amount effective to stimulate CL regression. In an embodiment, the cloprostenol sodium composition is estroPLAN®, comprising cloprostenol sodium, available from Parnell Technologies Pty Ltd, Alexandria, Australia. Each mL of the aqueous solution contains 263 mcg of cloprostenol sodium, 1.0 mcg of chlorocresol, 0.66 mg of citric acid anhydrous, 5.03 mg of sodium citrate 5.03, and 6.75 mg of sodium chloride. In an embodiment, each mL of the aqueous solution contains between about 1 mcg cloprostenol sodium to about 1000 mcg cloprostenol sodium.

The dinoprost tromethamine composition contains an analog of PGF2α in an amount effective to stimulate CL regression. In an embodiment, each mL of an aqueous solution of the dinoprost tromethamine composition contains between about 1 mg dinoprost tromethamine to about 50 mg dinoprost tromethamine.

The first application of the GnRH composition is administered in an amount effective to initiate the release of the gonadotropins FSH and LH from the anterior pituitary, thereby initiating a follicular wave. In an embodiment, the first application of the GnRH composition is a gonadorelin composition.

The first application of the PGF2α composition is administered in an amount effective to cause a functional and morphological regression of the CL (luteolysis). In an embodiment, the first application of the PGF2α composition is a cloprostenol sodium composition. In an embodiment, the first application of the PGF2α composition is a dinoprost tromethamine composition.

The second application of the PGF2α composition is administered in an amount effective to ensure complete luteal regression. In an embodiment, the second application of the PGF2α composition is a cloprostenol sodium composition. In an embodiment, the second application of the PGF2α composition is a dinoprost tromethamine composition.

The second application of the GnRH composition is administered in an amount effective to induce a pre-ovulatory surge in LH in a predictable manner, thereby inducing the animal to ovulate a dominant follicle in a specified timeframe thereby allowing FTAI. In an embodiment, the second application of the GnRH composition is a gonadorelin composition.

### EXPERIMENTS

Experiments were conducted to determine the effect of a second treatment of a PGF2α analog, in particular, a cloprostenol sodium composition, during an Ovsynch protocol on the regression of the CL, as measured by circulating P4, and on fertility to the FTAI. Two experiments were performed. In both experiments, cows were randomized to receive: 1) no additional treatments of cloprostenol sodium composition; and 2) a second treatment of cloprostenol sodium composition after the first treatment of the cloprostenol sodium composition.

### Experiment 1

An experiment was conducted to determine the effectiveness of the exogenous administration of hormones for synchronizing the estrous cycles of lactating dairy cattle and resulting pregnancy rates subsequent to FTAI. A total of 373 lactating Holstein cows (172 primiparous, 201 multiparous) were synchronized with a Double-Ovsynch program comprising a first or presynchronization Ovsynch phase followed by a second Ovsynch phase. All animals were synchronized using a GnRH composition (about 100 mcg of gonadorelin acetate per mL, GONAbreed®, Parnell Technologies Pty Ltd, Alexandria, Australia) and a PGF2α analog composition (about 250 mcg of cloprostenol sodium per mL, estroPLAN®, Parnell Technologies Pty Ltd, Alexandria, Australia). The first Ovsynch phase began at about 51 to about 57 days in milk where the cows received a first 1 mL intramuscular injection of a gonadorelin acetate composition. At about 58 to about 64 days in milk, the cows received a first 2 mL intramuscular injection of a cloprostenol sodium composition. At about 61 to about 67 days in milk, the cows received a second 1 mL intramuscular injection of a gonadorelin acetate composition concluding the first Ovsynch phase. The animals were then synchronized with a second Ovsynch phase comprising an Ovsynch-56 protocol beginning at about 68 to about 74 days in milk where the cows received a third 1 mL intramuscular injection of gonadorelin acetate composition. At about 75 to about 81 days in milk, the animals received a second 2 mL intramuscular injection of a cloprostenol sodium composition. At about 77 to about 83 days in milk (about 56 hours after the second injection of a cloprostenol sodium composition), the animals received a fourth 1 mL intramuscular injection of a gonadorelin acetate composition. And at about 78 to about 84 days in milk (about 16 hours after the fourth injection of the gonadorelin acetate composition), the animals receive AI, concluding the second Ovsynch phase.

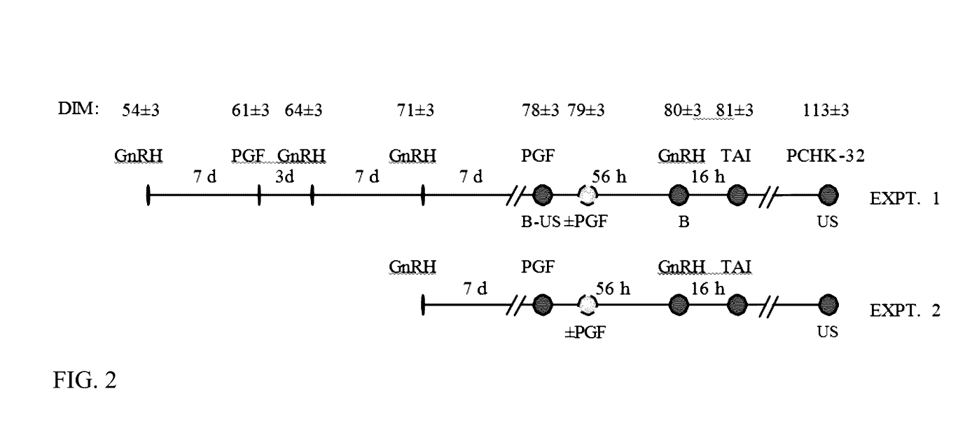

At the time of the second injection of the cloprostenol sodium composition during the double Ovsynch program (first cloprostenol injection during the second Ovsynch phase) the animals were randomized to one of two treatment groups: 1) a control group that did not receive any additional injections of a cloprostenol sodium composition; and 2) a treatment group that received a second 2 mL intramuscular injection of a cloprostenol sodium composition at about 77 to about 83 days in milk (about 24 hours after the first injection of the cloprostenol sodium composition during the second Ovsynch phase). The experimental protocol is shown in the diagram of FIG. 2.

Blood samples of the cows were taken at the time of the second injection of the cloprostenol sodium composition (at about 72 hours before FTAI) and at the time of the fourth injection of the gonadorelin acetate composition (at about 16 hours before FTAI). Blood samples were collected from the coccygeal vessels into vacuum tubes. Following collection, the blood samples were placed on ice and transported to a laboratory. After clotting (about 16 hours), samples were centrifuged at 3,000 rpm for 20 minutes and serum was isolated into vials, frozen and stored at −20 Celsius until assayed for P4 concentrations. Progesterone was determined directly from serum using a solid phase radioimmunoassay kit (Coat-A-Count, Siemens Healthcare Diagnostics, Los Angeles, Calif.) with no extraction. The assay had a sensitivity of 0.04 ng/mL and a coefficient of variation of 2.2%.

Statistical analyses were performed with SAS (SAS Institute, 2006). Variables with a binomial distribution, such as P/AI and percentage of animals with CL regression, were analyzed by logistic regression using the LOGISTIC procedure. One-tailed comparisons were utilized throughout the study for comparison of the effect of an additional cloprostenol sodium injection on CL regression and animals pregnant to the FTAI. Continuous variables were analyzed using ANOVA with logistic regression utilized to compare the relationship between circulating P4 and the binomial traits. Animals with ≦2.0 ng/mL P4 on day of PGF were removed from the analysis (n=29; 13 primiparous, 16 multiparous) due to lack of synchronization to the double Ovsynch protocol. Therefore, all analyses were done with a total of 344 lactating dairy cows (n=159 primiparous; n=185 multiparous). Statistical differences were considered significant for P≦0.05 and as a tendency for P≦0.15.

Circulating P4 concentrations during the second Ovsynch phase on the day of the first injection of a cloprostenol sodium composition were similar for cows receiving a single injection of a cloprostenol sodium composition (7.0+/−0.21; n=176) and cows receiving two injections of a cloprostenol sodium composition (7.1+/−0.20; n=168). Overall primiparous cows had greater circulating P4 concentrations than multiparous cows (7.7+/−0.22 vs. 6.5+/−0.18; P=0.0001). Treatment with either one or two injections of a cloprostenol sodium composition resulted in reduced circulating P4 concentrations in all cows in both treatments. During the second Ovsynch phase, the circulating P4 concentrations at 56 hours after the first injection of a cloprostenol sodium composition (the day of the of final injection of the gonadorelin acetate composition) were greater (P=0.005) for cows that received a single injection of the cloprostenol sodium composition (0.4+0.04) compared to two injections of the cloprostenol sodium composition (0.2+0.05).

The percentage of cows with complete CL regression (<0.5 ng/mL at 56 hours after the first injection of a cloprostenol sodium composition) is shown in Table 1.

TABLE 1
Effect of one vs. two treatments with a cloprostenol sodium
composition on percentage of cows with complete regression
of the corpus luteum in primiparous and multiparous cows
synchronized with a double Ovsynch protocol (Experiment 1)

| % of cows with | | | |
|---|---|---|---|
| complete | One cloprostenol | Two cloprostenol | |
| CL regression (n/n) | sodium injection | sodium injections | P-value |
| Primiparous | 81.2% (65/80) | 97.5% (77/79) | 0.001 |
| Multiparous | 84.4% (81/96) | 96.7% (86/89) | 0.006 |
| P-value | 0.69 | 1.0 | |
| Overall | 83.0% (146/176) | 97.0% (163/168) | 0.0001 |

Overall there were no differences between parities in the percentage of cows with complete CL regression. There were more cows with CL regression following two compared to one treatment of the cloprostenol sodium composition. This treatment effect on CL regression was observed when all cows were used in the analysis (P=0.0001) or when only primiparous (P=0.001) or multiparous (P=0.006) cows were used in the analysis.

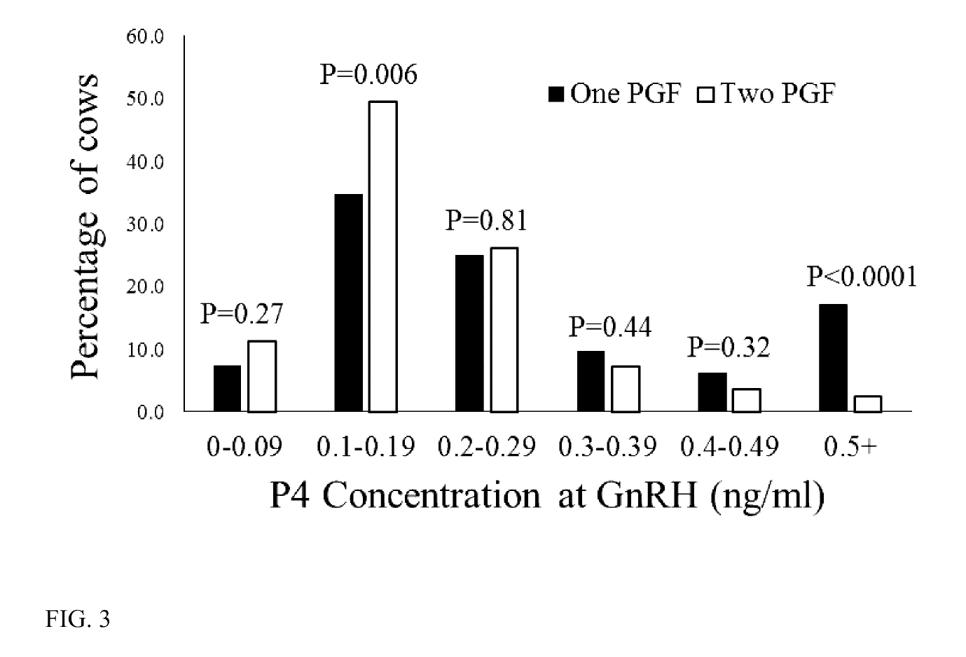

The distribution of cows based on circulating P4 at the time of the final injection of gonadorelin acetate composition is shown in FIG. 3. Overall, there were a greater (P<0.0001) percentage of cows with higher P4 (>0.5 ng/mL) in cows receiving one injection of a cloprostenol sodium composition (17.0%; 30/176) than two injections of a cloprostenol sodium composition (2.4%; 4/168). Conversely, there were a greater (P=0.006) percentage of cows with lower P4 (0.1 to 0.19) for the two cloprostenol sodium treatment group (49.4%; 83/168) than the one cloprostenol sodium treatment group (34.7%; 61/176).

If the comparison utilized all cows below 0.2 ng/mL, there is also a greater (P=0006) percentage of cows with lower P4 following treatment with two injections of a cloprostenol sodium composition (60.7%; 102/168) compared to treatment with one injection of a cloprostenol sodium composition (42.0%; 74/176).

Overall there were no statistically significant treatment differences in P/AI (Table 2).

TABLE 2
Effect of one vs. two treatments with PGF on percentage of
cows pregnant/AI (P/AI) in primiparous and multiparous cows
synchronized with Double-Ovsynch (Experiment 1). The relative
difference between treatments is calculated as the difference
in P/AI between two PGF minus one PGF treatments and then
divided by the one PGF P/AI results.

| | One cloprostenol | Two cloprostenol | Effect of PGF |
|---|---|---|---|
| | sodium injection | sodium injections | Difference (P-value) |
| Primiparous | 45.0% (36/80) | 48.2% (39/79) | 7.11% (P = 0.35) |
| Multiparous | 37.5% (36/96) | 44.6% (41/89) | 18.9% (P = 0.15) |
| P-value | 0.36 | 0.76 | |
| Overall | 40.1% (72/176) | 46.2% (80/168) | 15.2% (P = 0.13) |

Nevertheless, there was a tendency for an effect of two injections of a cloprostenol sodium composition to have greater P/AI than one injection of a cloprostenol sodium composition for multiparous cows (P=0.15) and for all cows (P=0.13). There were no statistical differences in P/AI between parities for cows receiving one injection of a cloprostenol sodium composition (P=0.36), two injections of a cloprostenol sodium composition (P=0.76), or for all cows (P=0.33).

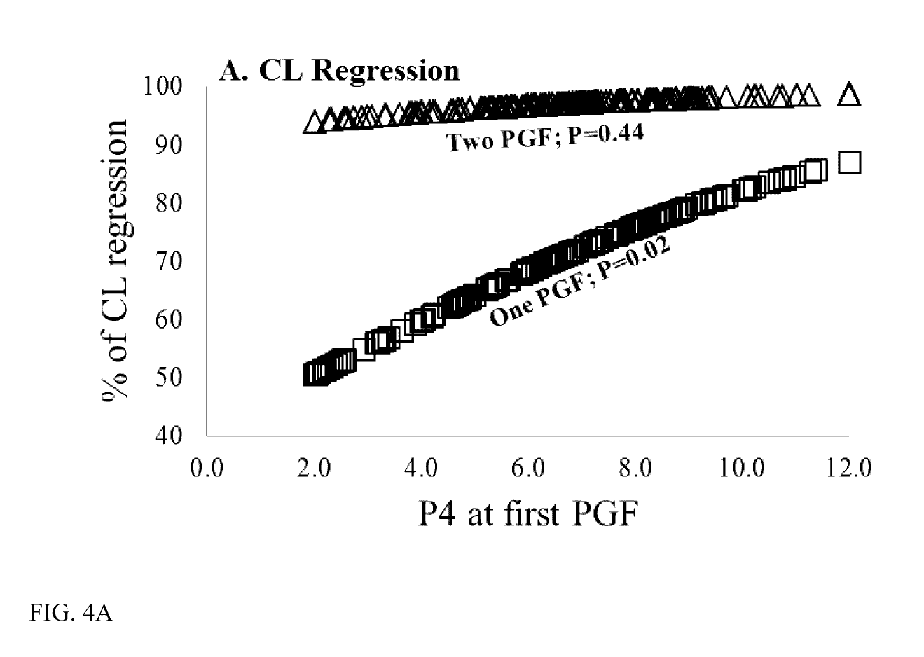

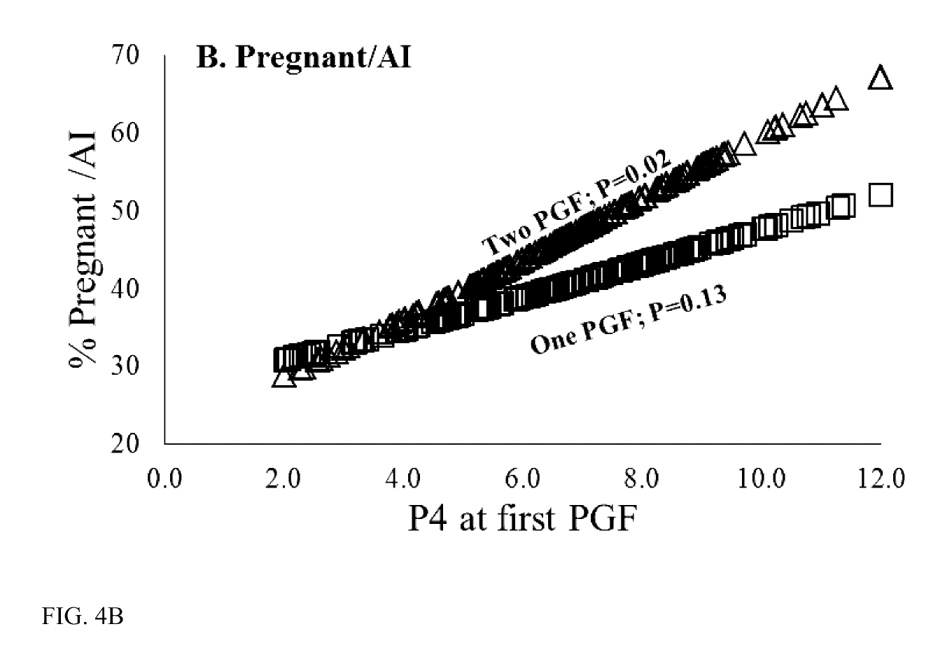

FIG. 4 shows the relationships between circulating P4 concentrations at the time of the first injection of the cloprostenol sodium composition and the probability of CL regression and P/AI in cows that received one injection compared to two injections. For CL regression (FIG. 4A), cows with lower P4 at the time of the first injection had a reduced percentage of cows with complete CL regression for cows receiving only one injection (P=0.02). In contrast, for cows receiving two injections of the cloprostenol sodium composition, there was no effect (P=0.44) of circulating P4 at the time of injection on percentage of cows with CL regression with almost all cows having complete CL regression, regardless of circulating P4 at time of the first injection.

FIG. 4B shows that both groups of cows had the lowest P/AI in cows that had lower P4 at the first injection. For cows receiving a single injection of a cloprostenol sodium composition, there was a tendency (P=0.13) for increasing P/AI in cows with increasing P4 at the time of injection. For cows receiving two injections of a cloprostenol sodium composition there was greater (P=0.02) P/AI with increasing circulating P4 at the time of injection. In addition, combining all cows (one or two injections), there is increasing P/AI with increasing circulating P4 at the time of the first injection of a cloprostenol sodium composition (data not shown; P=0.01).

The relationship of P4 at the first injection of a cloprostenol sodium composition with percentage of cows with complete CL regression could be further illustrated by doing a quartile analysis of cows, based on circulating P4 at the time of the first injection of a cloprostenol sodium composition (Table 3).

TABLE 3
Quartile analysis based on circulating P4 concentration [P4]
at time of first cloprostenol sodium injection. The percentage
of cows with CL regression and pregnant/AI are shown for cows
treated with either one or two cloprostenol sodium injection
injections in Experiment 1.

| | Quartile 1 | Quartile 2 | Quartile 3 | Quartile 4 |
|---|---|---|---|---|
| One | | | | |
| cloprostenol | | | | |
| sodium | | | | |
| injection | | | | |
| [P4] (range) | 2.0-4.8 | 4.9-6.7 | 6.8-8.7 | 8.8-12 |
| CL regression, | 66.7 (28/42) | 84.4 (38/45) | 95.5 (42/44) | 84.4 (38/45) |
| % (n/n) | | | | |
| Pregnant/AI, % | 31.0 (13/42) | 40.0 (18/45) | 47.7 (21/44) | 44.4 (20/45) |
| (n/n) | | | | |
| Two | | | | |
| cloprostenol | | | | |
| sodium | | | | |
| injections | | | | |
| [P4] (range) | 2.0-5.4 | 5.5-7.0 | 7.1-8.6 | 8.7-12 |
| CL regression, | 95.1 (39/41) | 95.6 (43/45) | 100 (43/43) | 97.4 (38/39) |
| % (n/n) | | | | |
| Pregnant/AI, % | 31.7 (13/41) | 46.7 (21/45) | 55.8 (24/43) | 56.4 (22/39) |
| (n/n) | | | | |

For cows receiving a single injection of a cloprostenol sodium composition, cows in Quartile 1 (lowest P4 concentrations; 2.0 to 4.8 ng/mL) had lower (P=0.0016) CL regression (66.7%; 28/42) compared to the other 3 quartiles (88.1%; 118/134) which did not differ from each other. In contrast in cows receiving two injections of a cloprostenol sodium composition, Quartile 1 cows (95.1%; 39/41) had similar (P=0.60) CL regression as observed for cows in the other three quartiles receiving two injections (97.6%; 124/127). Further, a comparison between cows receiving one vs. two injections within quartiles demonstrated that cows receiving one injection had lower CL regression receiving two injections, for cows in Quartile 1 (66.7% vs. 95.1%; P=0.0016) or for cows in the combination of quartiles 2, 3, and 4 (88.1% vs. 97.6%; P=0.0034).

Quartile analysis also demonstrated that cows with lower P4 at the time of injection (Quartile 1) had similar P/AI for cows receiving one (31.0%; 13/42) compared to two (31.7%; 13/41) injections (Table 3). In contrast for Quartiles 2, 3, and 4 there was a tendency (P=0.10) for decreased P/AI in cows receiving one (44.0%; 59/134) compared to two (52.8%; 67/127) injections. This numerical difference between one and two injections of a cloprostenol sodium composition could be observed in Quartile 2 (40.0% vs. 46.7%; P=0.34), Quartile 3 (47.7% vs. 55.8%; P=0.30), and Quartile 4 (44.4 vs. 56.4%; P=0.19).

In Experiment 1, treatment with a second PGF increased the percentage of cows with complete CL regression at the end of the Double-Ovsynch protocol from 83% to 97%. Numerous other studies have also reported that Ovsynch-type protocols that utilize only a single PGF2α treatment have 10-25% of cows with inadequate regression of the CL near the time of final GnRH or AI. For example, in cows resynchronized at 32 days after TAI with Ovsynch (Resynch-32) only 83.7% of cows had complete CL regression (<0.4 ng/mL at 56 hours after PGF2α), whereas cows resynchronized with Double-Ovsynch had 89.1% CL regression.¹² In that study, the presence of a new CL had a major effect on whether the cows underwent complete CL regression since CL regression happened in 93% of cows without ovulation to the first GnRH, but in only 79.4% of cows that ovulated to the first GnRH.¹² A study comparing dinoprost (25 mg) and cloprostenol (0.5 mg) reported similar (80% vs. 79%, respectively) percentage of cows with complete CL regression for the two products.¹⁵ Similarly, other studies using the 7d Ovsynch protocol have reported between 73.9% to 95.2% of cows with complete CL regression following a single treatment with different types of PGF products in lactating dairy cows.^{11, 25, 27} In our study, 18% of cows did not completely regress the CL after a single PGF treatment and, in contrast to previous studies^{10, 15} inadequate CL regression was similar in primiparous and multiparous cows. Further, we not only found a decrease in percentage of cows with elevated P4 (>0.5 ng/mL) but we also found a change in the distribution of cows by circulating P4 at final GnRH with an increased percentage of cows with very low circulating P4 (<0.2 ng/mL) after two compared to one PGF treatment. What was particularly intriguing in our analysis was that inadequate CL regression occurred in a larger percentage (33.3%) of cows with lower P4 concentrations (2.0 to 4.8 ng/mL) at the time of the single PGF treatment, compared to cows with greater circulating P4 at time of PGF (11.9%). Treatment with two PGFs produced complete CL regression in almost all cows, whether they had lower P4 (95.1%) or elevated P4 (97.6%) at time of first PGF treatment. It should be noted that we eliminated non-synchronized cows (7.8%; 29/373), by using a limit of 2 ng/ml of circulating P4 at the PGF treatment. A previous study also reported that cows with low P4 at PGF were more likely to not have complete CL regression.¹⁵ This previous study did not eliminate cows that were below 2 ng/mL at the time of PGF, used both cloprostenol or dinoprost as the PGF source, and measured P4 on three separate occasions to determine complete CL regression (56, 72, and 96 h after PGF). Based on results from both these studies, it seem clear that the largest problem with complete CL regression is due to cows that have low P4 at the time of PGF treatment, although even cows with higher P4 have more than 10% of cows with inadequate CL regression (Table 3). The reason that cows with lower P4 have a relative resistance to PGF action is not clear at this time. In a previous study¹² cows treated with PGF were much more likely to not have complete CL regression, if cows had a single d7 CL (35.6%) than if they had a single d14 CL (3.0%), or a d7 and a d14 CL (8.2%). Thus, regression of the d14 CL may be enhancing regression of the d7 CL, possibly due to a more pronounced decline in circulating P4 and resulting release of PGF from the uterus. Cows with lower P4 in our study or in the previous study¹⁵ may not have had as large of a decline in circulating P4 and consequently no augmentation of luteolysis from the uterus. Definitive testing of this speculative idea or other potential reasons for lack of complete CL regression will require future research.

Experiment 1 was not powered for detection of fertility effects due to the second PGF, however there was a tendency for an improvement in fertility when all cows were analyzed (P=0.13) or if only multiparous cows were evaluated (P=0.15). For cows given either one or two PGF treatments, there was increasing fertility with increasing circulating P4 at the time of PGF treatment. This relationship has been previously reported for cows given only a single PGF treatment.¹⁵ However, in our study, this relationship was particularly clear in the cows given two PGF treatments (P=0.02) and this has not been previously reported. Of particular interest, the second PGF only appeared to enhance fertility in cows with elevated P4 and not in cows that had low P4 at the first PGF treatment. This is puzzling because the greatest improvement in CL regression caused by the second PGF treatment was found in the cows with low P4 at the time of PGF treatment. This intriguing result will need to be replicated in future studies but suggests that the second PGF may be enhancing fertility by effects other than decreasing percentage of cows with inadequate luteolysis at 56 h after PGF. Perhaps an earlier time to low P4, increased circulating estradiol, or other effects of a second PGF treatment underlie any observed improvements in fertility produced by a second PGF treatment.

### Experiment 2

An experiment was performed on 11 commercial dairies in different locations in the United States: 3 dairies in Wisconsin; 3 dairies in California; 2 dairies in New York; 1 dairy in Pennsylvania; 1 dairy in New Mexico; and 1 dairy in Texas. Although overall management was quite different for each herd, each herd used the same reproductive management protocol during this experiment. Cows were randomly assigned within herd, parity, and date to one of three treatment groups at about 40 to about 46 days in milk. One of the treatment groups, Presynch-Ovsynch with heat detection, was not considered in the present analysis. Therefore only two treatment groups were utilized for all analyses, as shown in the diagram for Experiment 2 in FIG. 2.

All animals were synchronized using a GnRH composition (about 100 mcg of gonadorelin acetate per mL, GONAbreed®, Parnell Technologies Pty Ltd, Alexandria, Australia) and a PGF2α analog composition (about 250 mcg of cloprostenol sodium per mL, estroPLAN®, Parnell Technologies Pty Ltd, Alexandria, Australia).

One treatment group began at about 47 to about 53 days in milk where the cows received a first 1 mL intramuscular injection of a gonadorelin acetate composition. At about 54 to about 60 days in milk, the cows received a first 2 mL intramuscular injection of a cloprostenol sodium composition. At about 56 to about 62 days in milk (about 56 hours after the first injection of the cloprostenol sodium composition), the cows received a second 1 mL intramuscular injection of a gonadorelin acetate composition. And at about 57 to about 63 days in milk (about 16 hours after the second injection of the gonadorelin acetate composition), the animals received AI, concluding the Ovsynch phase.

The other treatment group began at about 47 to about 53 days in milk where the cows received a first 1 mL intramuscular injection of a gonadorelin acetate composition. At about 54 to about 60 days in milk, the cows received a first 2 mL intramuscular injection of a cloprostenol sodium composition. At about 55 to about 61 days in milk (about 24 hours after the first injection of the cloprostenol sodium composition), the cows received a second 2 mL intramuscular injection of a cloprostenol sodium composition. At about 56 to about 62 days in milk (about 56 hours after the first injection of the cloprostenol sodium composition), the cows received a second 1 mL intramuscular injection of a gonadorelin acetate composition. And at about 57 to about 63 days in milk (about 16 hours after the second injection of the gonadorelin acetate composition), the animals received AI, concluding the Ovsynch phase.

Pregnancy diagnosis was performed with ultrasound utilizing the normal schedule for each herd (Wisconsin and Pennsylvania herds, 31-33 days after AI; California, New York, New Mexico, and Texas herds, 37-43 days after AI). Data on all treatments, AI, and pregnancy diagnosis were recorded for each individual cow in most cases (n=1,912). In some cows, data was extrapolated from other recorded data due to missing one treatment recording (n=110) or two or more treatment recordings in a cow (n=126). Pregnancy was based on a verified pregnancy diagnosis. Non-pregnancy was based on absence of pregnancy at a pregnancy diagnosis or a rebreeding to an estrus prior to pregnancy diagnosis. Statistical analyses were performed using PROC Logistic in SAS. Treatment and parity were forced into the model and factors were kept in the model if they were significant at P<0.15.

The results from all 11 farms are shown in Table 4.

TABLE 4
Results for effects of treatments on each individual farm in
Experiment 2. The relative difference between treatments is calculated as
the difference between Ovsynch + second cloprostenol sodium injection
minus Ovsynch and divided by the Ovsynch results. The P Values were
calculated using Chi Square analyses.

| | | | Ovsynch + second | | Effect of second | |
|---|---|---|---|---|---|---|
| | | | cloprostenol sodium | | cloprostenol sodium | |
| | Ovsynch | | injection | | injection Difference (P | |
| Farm | % (n/n) | | % (n/n) | | Value) | |
| CA-01 | 29.4 | (25/85) | 36.4 | (32/88) | +23.64% | (P = 0.209) |
| CA-02 | 35.3 | (43/122) | 39.8 | (45/113) | +12.99% | (P = 0.278) |
| CA-O3 | 30.4 | (38/125) | 34.1 | (47/138) | +12.03% | (P = 0.308) |
| NM-04 | 35.2 | (68/193) | 37.7 | (66/175) | +7.04% | (P = 0.350) |
| NY-05 | 32.7 | (32/98) | 31.2 | (34/109) | −4.47% | (P = 0.469) |
| NY-06 | 29.4 | (27/92) | 42.0 | (42/100) | +43.11% | (P = 0.01) |
| PA-07 | 39.2 | (31/79) | 43.6 | (27/62) | +10.98% | (P = 0.365) |
| TX-08 | 37.7 | (46/122) | 29.8 | (37/124) | −20.86% | (P = 0.121) |
| WI-09 | 28.1 | (16/57) | 40.4 | (23/57) | +43.75% | (P = 0.118) |
| WI-10 | 35.3 | (18/51) | 37.0 | (20/54) | −4.94% | (P = 0.507) |
| WI-11 | 26.9 | (14/52) | 26.9 | (14/52) | 0.0% | (P = 1.0) |
| Total | 33.3% | (358/1076) | 36.1% | (387/1072) | +8.50% | (P = 0.068) |

Each of the farms had at least 100 cows assigned to the experiment. One of the farms (New York—06) had a significant positive effect of treatment on results, while one farm (Wisconsin—09) had a tendency for a positive effect and one farm (Texas—08) had a tendency for a negative effect of treatment. Overall, there was a tendency (P=0.068) for an effect of treatment on P/AI with an increase from 33.3% in Ovsynch compared to 36.1% in Ovsynch+a second cloprostenol sodium injection.

In the overall logistic regression model there was no significant effect of farm on the results (P=0.712). There was also no interaction of parity and treatment in the overall model (P=0.741). Therefore these two factors were removed from the final logistic model. There was a significant effect of parity in the final logistic model (P=0.03) and there was a tendency for an effect of treatment (0.0776). The results, divided by parity are shown in Table 5.

TABLE 5
Results of Experiment 2 for effects of treatment and
parity on percentage of cows pregnant per AI.

| | | Ovsynch + second | Effect of second |
|---|---|---|---|
| | | cloprostenol sodium | cloprostenol |
| | Ovsynch | injection | sodium injection |
| Parity | % (n/n) | % (n/n) | Difference (P Value) |
| Primiparous | 37.07 (99/267) | 38.22 (99/259) | +3.10% (P = 0.393) |
| All Multiparous | 32.01 (259/809) | 35.42 (288/813) | +10.65% (P = 0.073) |
| P Value | 0.128 | 0.414 | |
| Second and Third | 32.71 (194/593) | 37.35 (220/589) | +14.19% (P = 0.047) |
| Fourth or Greater | 30.09 (65/216) | 30.36 (68/224) | +0.09% (P = 0.517) |

There was no effect of treatment in primiparous cows (P=0.393). However, there was a tendency for an effect of treatment in multiparous cows (P=0.073). When multiparous cows were evaluated by parity, it was found that there was an effect of treatment in cows of second and third lactation (P=0.047) but no effect of treatment in older cows (fourth or greater lactations).

Experiment 1 and Experiment 2 were done with a similar experimental design and therefore results were combined in order to more thoroughly test the effect of a second cloprostenol sodium injection in the Ovsynch protocol on P/AI. There was no effect of the second PGF on P/AI in primiparous cows (P=0.304). However in multiparous cows, the second cloprostenol sodium injection increased (P=0.046) P/AI by about 12%. This effect of a second cloprostenol sodium injection on P/AI was also observed when all cows were included in the analysis (P=0.042).

Experiment 2 represents one of the larger multi-site studies undertaken to examine different reproductive management protocols. Eleven different herds were found in the West, Southwest, Midwest, and Northeast regions of the USA. There was no detectable effect of farm found from the logistic regression analysis, although, as shown in Table 4, treatment effects ranged from a significant positive effect (P=0.01) to a tendency for a negative effect (P=0.12) on individual farms. When all results were combined there was about a 3% absolute improvement or an 8.5% relative improvement in fertility due to the second PGF treatment. Nonetheless, even with over 2,100 cows in this experiment, the fertility enhancement was only a statistical tendency (P=0.068). As in experiment 1, the fertility enhancement was only observed in multiparous (P=0.073) and not in primiparous (P=0.39) cows. Further separation of the parity groups showed that the fertility enhancement was only present in second and third lactation cows (P=0.047) and not in fourth or greater lactation cows (P=0.517). We can think of no reasonable physiological explanation for this odd parity effect and assume that the lack of effect in the oldest cows is just a numerical aberration. Greater fertility in primiparous than multiparous cows has been previously reported in numerous studies using Ovsynch-type protocols.^{10, 13, 32}

The final combined analysis of both experiments provided the most statistical power to test our main hypothesis (Table 6). Overall, there was increased (P=0.042) P/AI with a 9.6% relative increase in fertility. As in both experiments, the effect was only statistically detectable in multiparous (P=0.046) and not in primiparous (P=0.304) cows. In our study an effect of parity on fertility was only detected in cows treated with Ovsynch (P=0.04) and not in cows treated with Ovsynch with the second PGF treatment (P=0.17). Future studies should directly test whether parity effects with Ovsynch-type protocols are related primarily to differences in CL regression between parities or reflect other fertility differences related to parity.

From a practical standpoint, the improvement in fertility produced by a second PGF treatment, about 10% more pregnancies following the Ovsynch protocol, is likely to make this treatment economically beneficial for many dairy farms. The costs for a single PGF are likely to be more than offset by the reduction in semen costs, reduction in future costs for detection of estrus or for synchronization of non-pregnant cows, and increased value from the earlier pregnancy.⁸ The value of a second PGF is clearly greater for multiparous than primiparous cows and therefore these cows should be targeted with this extra PGF treatment. The results that were observed using the circulating P4 concentrations indicate that future research may allow even more precise targeting of cows that are most likely to benefit from the second PGF treatment.

Full citations for the documents referred to in the specification:
1. Ayres, H., R. M. Ferreira, A. P. Cunha, R. R. Araujo, and M. C. Wiltbank. 2013. Double-Ovsynch in high-producing dairy cows: Effects on progesterone concentrations and ovulation to GnRH treatments. Theriogenology 79:159-164.
2. Bello, N. M., J. P. Steibel, and J. R. Pursley. 2006. Optimizing ovulation to first GnRH improved outcomes to each hormonal injection of Ovsynch in lactating dairy cows. J. Dairy Sci. 89:3413-3424.
3. Bisinotto, R. S., R. C. Chebel, and J. E. Santos. 2010. Follicular wave of the ovulatory follicle and not cyclic status influences fertility of dairy cows. J Dairy Sci 93:3578-3587.
4. Brusveen, D. J., A. H. Souza, and M. C. Wiltbank. 2009. Effects of additional prostaglandin F-2 alpha and estradiol-17 beta during Ovsynch in lactating dairy cows. J. Dairy Sci. 92:1412-1422.
5. Chebel, R. C., J. E. Santos, R. L. Cerri, H. M. Rutigliano, and R. G. Bruno. 2006. Reproduction in dairy cows following progesterone insert presynchronization and resynchronization protocols. J Dairy Sci 89:4205-4219.
6. Ei-Zarkouny, S. Z., J. A. Cartmill, B. A. Hensley, and J. S. Stevenson. 2004. Pregnancy in dairy cows after synchronized ovulation regimens with or without presynchronization and progesterone. J. Dairy Sci. 87:1024-1037.
7. Galvao, K. N., M. F. Sa Filho, and J. E. Santos. 2007. Reducing the interval from presynchronization to initiation of timed artificial insemination improves fertility in dairy cows. J Dairy Sci 90:4212-4218.
8. Giordano, J. O., P. M. Fricke, M. C. Wiltbank, and V. E. Cabrera. 2011. An economic decision-making support system for selection of reproductive management programs on dairy farms. J. Dairy Sci. 94:6216-6232.
9. Giordano, J. O., J. N. Guenther, M. S. Ares, M. C. Wiltbank, and P. M. Fricke. 2010. Presynchronization with hCG 7 d before initiation of Resynch improves fertility similar to a double-Ovsynch Resynch protocol in lactating dairy cows. J. Dairy Sci. 93:219-219.
10. Giordano, J. O., M. C. Wiltbank, P. M. Fricke, S. Bas, R. A. Pawlisch, J. N. Guenther, and A. B. Nascimento. 2013. Effect of increasing GnRH and PGF2α dose during Double-Ovsynch on ovulatory response, luteal regression, and fertility of lactating dairy cows. Theriogenology 80:773-783.
11. Giordano, J. O., M. C. Wiltbank, J. N. Guenther, M. S. Ares, G. Lopes, Jr., M. M. Herlihy, and P. M. Fricke. 2012a. Effect of presynchronization with human chorionic gonadotropin or gonadotropin-releasing hormone 7 days before resynchronization of ovulation on fertility in lactating dairy cows. J Dairy Sci 95:5612-5625.
12. Giordano, J. O., M. C. Wiltbank, J. N. Guenther, R. Pawlisch, S. Bas, A. P. Cunha, and P. M. Fricke. 2012b. Increased fertility in lactating dairy cows resynchronized with Double-Ovsynch compared with Ovsynch initiated 32 d after timed artificial insemination. J. Dairy Sci. 95:639-653.
13. Herlihy, M. M., J. O. Giordano, A. H. Souza, H. Ayres, R. M. Ferreira, A. Keskin, A. B. Nascimento, J. N. Guenther, J. M. Gaska, S. J. Kacuba, M. A. Crowe, S. T. Butler, and M. C. Wiltbank. 2012. Presynchronization with Double-Ovsynch improves fertility at first postpartum artificial insemination in lactating dairy cows. J Dairy Sci 95:7003-7014.
14. Keskin, A., G. Yilmazbas-Mecitoglu, A. Gumen, E. Karakaya, R. Darici, and H. Okut. 2010. Effect of hCG vs. GnRH at the beginning of the Ovsynch on first ovulation and conception rates in cyclic lactating dairy cows. Theriogenology 74:602-607.
15. Martins, J. P., R. K. Policelli, L. M. Neuder, W. Raphael, and J. R. Pursley. 2011. Effects of cloprostenol sodium at final prostaglandin F2alpha of Ovsynch on complete luteolysis and pregnancy per artificial insemination in lactating dairy cows. J Dairy Sci 94:2815-2824.
16. Moreira, F., C. Orlandi, C. A. Risco, R. Mattos, F. Lopes, and W. W. Thatcher. 2001. Effects of presynchronization and bovine somatotropin on pregnancy rates to a timed artificial insemination protocol in lactating dairy cows. J. Dairy Sci. 84:1646-1659.
17. Nascimento, A. B., A. H. Souza, A. Keskin, R. Sartori, and M. C. Wiltbank. 2014. Lack of complete regression of the Day 5 corpus luteum after one or two doses of PGF(2 alpha) in nonlactating Holstein cows. Theriogenology 81:389-395.
18. Navanukraw, C., D. A. Redmer, L. P. Reynolds, J. D. Kirsch, A. T. Grazul-Bilska, and P. M. Fricke. 2004. A modified presynchronization protocol improves fertility to timed artificial insemination in lactating dairy cows. J. Dairy Sci. 87:1551-1557.
19. Norman, H. D., J. R. Wright, S. M. Hubbard, R. H. Miller, and J. L. Hutchison. 2009. Reproductive status of Holstein and Jersey cows in the United States. J. Dairy Sci. 92:3517-3528.
20. Peters, M. W. and J. R. Pursley. 2002. Fertility of lactating dairy cows treated with Ovsynch after presynchronization injections of PGF(2 alpha) and GnRH. J. Dairy Sci. 85:2403-2406.
21. Pursley, J. R., M. R. Kosorok, and M. C. Wiltbank. 1997. Reproductive management of lactating dairy cows using synchronization of ovulation. J. Dairy Sci. 80:301-306.
22. Pursley, J. R., M. O. Mee, and M. C. Wiltbank. 1995. Synchronization of ovulation in dairy cows using PGF2alpha and GnRH. Theriogenology 44:915-923.
23. Rabiee, A. R., I. J. Lean, and M. A. Stevenson. 2005. Efficacy of Ovsynch program on reproductive performance in dairy cattle: A meta-analysis. J. Dairy Sci. 88:2754-2770.
24. Ribeiro, E. S., R. S. Bisinotto, M. G. Favoreto, L. T. Martins, R. L. A. Cerri, F. T. Silvestre, L. F. Greco, W. W. Thatcher, and J. E. P. Santos. 2012. Fertility in dairy cows following presynchronization and administering twice the luteolytic dose of prostaglandin F-2 alpha as one or two injections in the 5-day timed artificial insemination protocol. Theriogenology 78:273-284.
25. Santos, J. E. P., C. D. Narciso, F. Rivera, W. W. Thatcher, and R. C. Chebel. 2010. Effect of reducing the period of follicle dominance in a timed artificial insemination protocol on reproduction of dairy cows. J. Dairy Sci. 93:2976-2988.
26. Souza, A. H., H. Ayres, R. M. Ferreira, and M. C. Wiltbank. 2008. A new presynchronization system (Double-Ovsynch) increases fertility at first postpartum timed AI in lactating dairy cows. Theriogenology 70:208-215.
27. Souza, A. H., A. Gumen, E. P. B. Silva, A. P. Cunha, J. N. Guenther, C. M. Peto, D. Z. Caraviello, and M. C. Wiltbank. 2007. Supplementation with estradiol-17 beta before the last gonadotropin-releasing hormone injection of the Ovsynch protocol in lactating dairy cows. J. Dairy Sci. 90:4623-4634.
28. Thatcher, W. W., T. R. Bilby, J. A. Bartolome, F. Silvestre, C. R. Staples, and J. E. Santos. 2006. Strategies for improving fertility in the modern dairy cow. Theriogenology 65:30-44.
29. Thatcher, W. W., F. Moreira, S. M. Pancarci, J. A. Bartolome, and J. E. Santos. 2002. Strategies to optimize reproductive efficiency by regulation of ovarian function. Domest Anim Endocrinol 23:243-254.
30. Vasconcelos, J. L. M., R. W. Silcox, G. J. M. Rosa, J. R. Pursley, and M. C. Wiltbank. 1999. Synchronization rate, size of the ovulatory follicle, and pregnancy rate after synchronization of ovulation beginning on different days of the estrous cycle in lactating dairy cows. Theriogenology 52:1067-1078.
31. Wiltbank, M., H. Lopez, R. Sartori, S. Sangsritavong, and A. Gumen. 2006. Changes in reproductive physiology of lactating dairy cows due to elevated steroid metabolism. Theriogenology 65:17-29.
32. Wiltbank, M. C. and J. R. Pursley. 2014. The cow as an induced ovulator: Timed AI after synchronization of ovulation. Theriogenology 81:170-185.
33. Wiltbank, M. C., A. H. Souza, P. A. Carvahlo, A. P. Cunha, J. O. Giordano, P. M. Fricke, G. M. Baez, and M. G. Diskin. 2014. Physiological and practical effects of progesterone on reproduction in dairy cattle. Animal 8:s1:70-81.

Having thus described the disclosed subject matter, what is claimed as new and desired to be secured by Letters Patent is:

1. A method for synchronizing the estrous cycle in cattle, the steps comprising:
(a) administering a first dose of a GnRH composition to a lactating cow during an estrous cycle in an amount effective to stimulate a surge in the circulating concentrations of LH and FSH;
(b) subsequent to step (a) and after a first period of time sufficient for development of a dominant follicle and a functional corpus luteum, administering a first dose of a PGF2α composition in an amount effective to stimulate corpus luteum regression;
(c) subsequent to step (b) and after a second period of time sufficient for initiating the process of corpus luteum regression, administering a second dose of a PGF2α composition in an amount effective to effectuate complete corpus luteum regression;
(d) subsequent to step (c) and after a third period of time sufficient for complete luteal regression, administering a second dose of a GnRH composition in an amount effective to stimulate ovarian follicular development and subsequent ovulation; and
(e) subsequent to step (d) and after a fourth period of time, breeding the cow using artificial insemination.

2. The method of claim 1, wherein the first period of time is between about 144 hours and about 192 hours.

3. The method of claim 1, wherein the second period of time is between about 0 hours and about 56 hours.

4. The method of claim 1, wherein the third period of time is between about 0 hours and about 48 hours.

5. The method of claim 1, wherein the fourth period of time is between about 0 hours and about 24 hours.

6. The method of claim 1, wherein the fourth period of time is between about 8 hours and about 20 hours.

7. The method of claim 1, wherein the lactating cow is primiparous.

8. The method of claim 1, wherein the lactating cow is multiparous.

9. The method of claim 1, wherein breeding occurs during heat stress conditions.

10. The method of claim 1, wherein breeding occurs during non-heat stress conditions.

11. The method of claim 1, wherein the GnRH composition is a gonadorelin composition.

12. The method of claim 11, wherein the gonadorelin composition is gonadorelin acetate.

13. The method of claim 11, wherein the gonadorelin composition is gonadorelin hydrochloride.

14. The method of claim 1, wherein the PGF2α composition is a cloprostenol composition.

15. The method of claim 14, wherein the cloprostenol composition is cloprostenol sodium.

16. The method of claim 1, wherein the PGF2α composition is a dinoprost composition.

17. The method of claim 16, wherein the dinoprost composition is a dinoprost tromethamine composition.

18. The method of claim 1, wherein at the period of time subsequent to step (c) and before step (d) circulating P4 concentrations are less than at the period of time subsequent to step (b) and before step (c).
